(19) United States
(12) Patent Application Publication
(10) Pub. No.: US 2026/0241058 A1
(43) Pub. Date: Aug. 20, 2026

(54) Air Freshener With Detachable Collectible Portion

(71) Applicant: Sir Killian Wells, Austin, TX (US)

(72) Inventor: Sir Killian Wells, Austin, TX (US)

(73) Assignee: New Faction Inc d/b/a Xyrena, Austin, TX (US)

(21) Appl. No.: 19/445,591

(22) Filed: Jan. 11, 2026

Related U.S. Application Data
(60) Provisional application No. 63/760,592, filed on Feb. 19, 2025.

(51) Int. Cl. A61L 9/12; A61L 9/04; G06K 19/06; G09F 3/02; G09F 3/10

(52) U.S. Cl. CPC A61L 9/12; A61L 9/042; G06K 19/06028; G06K 19/06037; G09F 3/02; G09F 3/10; A61L 2209/15; G09F 2003/0226

(57) ABSTRACT

A dual-purpose fragrance and collectible article comprising a flat scented body divided into an upper fragrance-emitting portion and a lower detachable collectible portion by a frangible connection. Upon separation, the lower portion becomes a standalone collectible, transforming a consumable air freshener into a keepsake and enabling a collectible fragrance platform.

### CROSS-REFERENCE TO RELATED APPLICATIONS

This application claims the benefit of U.S. Provisional Patent Application No. 63/760,592, filed on Feb. 19, 2025, the entire contents of which are incorporated herein by reference.

### FIELD OF THE INVENTION

The present invention relates to fragrance-dispensing articles, and more particularly to air fresheners configured with detachable collectible portions, such as trading cards, stickers, postcards, or other memorabilia formats.

### BACKGROUND OF THE INVENTION

Flat air fresheners formed from scented cellulose or paperboard and suspended by elastic strings are widely known. Such products are consumable and discarded once fragrance is exhausted. Some prior products include perforated tear-off sections, but these sections have been limited to coupons, advertisements, or contact information and were not intended for long-term retention, display, or collection.

Separately, scented paper collectibles such as scratch-and-sniff cards, scented postcards, and novelty scented trading cards have existed, including character-based and franchise-based scented cards. However, these items function only as collectibles and do not serve as active fragrance-delivery devices when deployed in an environment.

Card-shaped scented novelty products have also appeared in the marketplace, including Pokémon-themed scented trading-card-style air fresheners marketed under names such as “Pokéscents.” Such products typically comprise a single, unitary scented card having printed artwork and, in some cases, a hanging string, but do not include a frangible connection dividing the article into an upper fragrance-delivery portion and a lower detachable collectible portion. These products therefore do not transition from a fragrance article into a standardized collectible upon detachment, and instead remain single-purpose novelty items throughout their lifecycle.

Thus, even when considering card-shaped scented novelty products such as Pokéscents, the prior art teaches either (a) air fresheners with incidental tear-off portions of no collectible value, or (b) single-piece scented collectibles that do not transition from a fragrance article into a standardized collectible via a frangible separation.

Accordingly, there exists a need for a fragrance article that transitions from a consumable air freshener into a durable collectible compatible with existing trading card, sticker, or memorabilia storage systems.

### SUMMARY OF THE INVENTION

The present invention provides a dual-purpose fragrance and collectible article comprising a unitary flat scented body divided into an upper portion and a lower portion by a frangible connection. The upper portion functions as the fragrance-emitting and display portion and may include a suspension feature and retail information. The lower portion is configured as a detachable collectible such as a trading card, sticker, postcard, or other memorabilia format.

Upon separation along the frangible connection, the lower portion becomes a standalone collectible free of hanging elements, tabs, or irregular edges. The collectible may retain fragrance after detachment or may be isolated from fragrance by a barrier layer.

This structure allows the product to serve first as an air freshener and then as a keepsake, extending product life, reducing waste, and creating a collectible fragrance ecosystem not contemplated by prior art.

Unexpectedly, users are incentivized to preserve and collect the detached lower portion, thereby transforming what would ordinarily be a disposable fragrance product into a long-term collectible and displayable item.

### BRIEF DESCRIPTION OF THE DRAWINGS

FIG. 1 is a front view of a first embodiment of the article showing an upper portion with a hanging element, a frangible connection, and a lower collectible portion.

FIG. 2 is a front view of an alternate embodiment having rounded corners and an upper hanging tab.

FIG. 3 is a front view of another alternate embodiment in which the upper portion is connected to the lower collectible portion by an elongated neck.

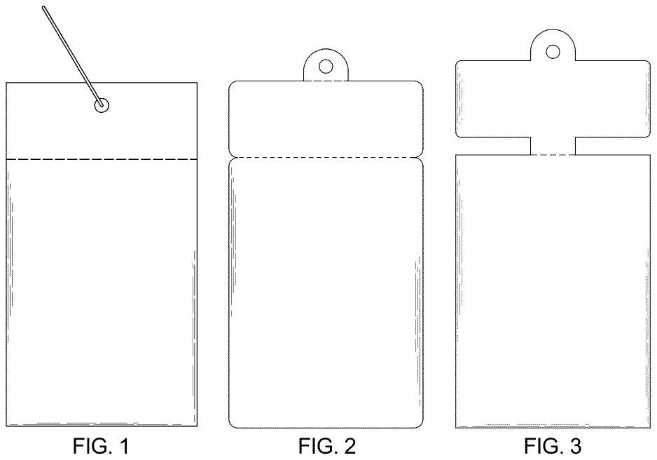

### DETAILED DESCRIPTION

The article comprises a substantially flat body formed from an absorbent substrate such as cellulose, paperboard, or absorbent cardstock, preferably having a thickness between 60 pt and 100 pt and more preferably approximately 80 pt. The body is preferably die-cut from a single sheet of material. Accordingly, the upper portion and the lower portion are initially formed as parts of a single unitary article prior to separation.

The body is divided into an upper portion and a lower portion by a frangible connection such as a perforation, micro-perforation, score line, kiss-cut, or weakened adhesive joint.

### Upper Portion

The upper portion is configured to emit fragrance and to be displayed or suspended. It may include a hole, slot, or removable tab for attachment of a hanging element such as an elastic string, cord, ribbon, or rigid hook. The upper portion may include printed product information such as fragrance name, brand, barcodes, QR codes, price codes, or series identifiers. A scannable code may provide access to digital collectible metadata, augmented-reality content, or series information.

### Lower Portion

The lower portion is configured as a detachable collectible such as a trading card, sticker, postcard, or other memorabilia. It may be double-sided and may include artwork, branding, or series information. Corners may be rounded. The lower portion may include adhesive backing and a release liner when configured as a sticker. The lower portion may be dimensioned for compatibility with standard trading card sleeves, binders, or rigid display cases.

### Fragrance

The substrate may be uniformly impregnated so that both portions are scented, or a barrier layer may prevent fragrance migration into the collectible portion.

### Packaging

The article may be packaged in a fragrance-impervious wrapper or pouch to preserve fragrance until opening.

1. A fragrance delivery article comprising: a single unitary flat body having an upper portion and a lower portion; a frangible connection between the upper portion and the lower portion configured to be separated by a user; the upper portion being configured to release fragrance and to be displayed or suspended; the lower portion being configured as a detachable standalone collectible intended for long-term collection or display; wherein separation along the frangible connection produces a standalone collectible.

2. The article of claim 1, wherein the flat body comprises cellulose, paperboard, or absorbent cardstock having a thickness between 60 pt and 100 pt.

3. The article of claim 1, wherein the flat body is die-cut from a single sheet of absorbent material.

4. The article of claim 1, wherein the frangible connection comprises a perforation, micro-perforation, score line, kiss-cut, or weakened adhesive joint.

5. The article of claim 1, wherein the lower portion is configured as a collectible selected from the group consisting of a trading card, a sticker, a postcard, or other memorabilia format.

6. The article of claim 1, wherein detachment results in the collectible having no residual tab, string, or protrusion beyond its perimeter.

7. The article of claim 1, wherein the upper portion includes a suspension feature selected from an elastic string, cord, ribbon, or rigid hook attached through a hole, slot, or removable tab.

8. The article of claim 7, wherein the suspension feature is attached to a removable or frangible tab such that detachment of the collectible also removes any hanging remnant.

9. The article of claim 1, wherein both the upper and lower portions are impregnated with fragrance.

10. The article of claim 1, wherein only the upper portion is impregnated with fragrance.

11. The article of claim 1, wherein the lower portion is double-sided with printed artwork.

12. The article of claim 1, wherein the lower portion has a height-to-width ratio between 1.3:1 and 1.5:1.

13. The article of claim 1, wherein the upper portion includes product-identifying information selected from a barcode, QR code, price code, fragrance name, or series identifier, such that the article functions as its own retail display package.

14. The article of claim 1, wherein the upper portion includes a machine-readable code scannable by a point-of-sale system prior to detachment of the collectible portion.

15. The article of claim 1, wherein the lower portion is dimensioned for compatibility with at least one of a trading card sleeve, binder, or rigid display case.

16. The article of claim 1, wherein the article is packaged in a fragrance-impervious wrapper or pouch.

17. The article of claim 1, wherein the scannable code provides access to digital collectible metadata or augmented-reality content.

18. The article of claim 5, wherein the collectible is a sticker having adhesive backing and a release liner.

19. The article of claim 5, wherein the collectible is a postcard.

20. The article of claim 1, wherein the lower portion includes rounded corners.

21. A method of providing fragrance and a collectible to a user, comprising: suspending a flat scented article having an upper portion and a detachable lower collectible portion; releasing fragrance from the upper portion; detaching the lower portion along a frangible connection; and retaining the lower portion as a standardized collectible for long-term collection or display.

22. A method of manufacturing a dual-purpose fragrance and collectible article, comprising:
impregnating a flat absorbent substrate with fragrance; printing artwork and retail information on at least one side; forming a frangible connection defining an upper portion and a lower portion;
forming a suspension feature in the upper portion; attaching a hanging element to the upper portion; and packaging the article to preserve fragrance.

23. The article of claim 1, wherein the lower portion includes a barrier layer configured to prevent fragrance oil migration from the upper portion into the collectible portion.

24. The article of claim 1, wherein the lower portion includes a serial number, authentication code, or unique identifier associated with the collectible.

25. The article of claim 1, wherein the lower portion is dimensioned to conform to a standardized collectible format.

26. A method of creating a collectible from a fragrance article, comprising: providing a flat scented article having an upper fragrance-delivery portion and a lower collectible portion; fracturing a frangible connection; and retaining the lower portion as a standardized collectible.

27. The article of claim 1, wherein the lower portion comprises a multilayer collectible including a non-paper insert or overlay.
